(19) United States
(12) Patent Application Publication
(10) Pub. No.: US 2007/0167876 A1
(43) Pub. Date: Jul. 19, 2007

(54) Occluding guidewire and methods

(76) Inventors: Charles Louis Euteneuer, St. Michael, MN (US); Duane G. Frion, Brooklyn Center, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/333,038

(22) Filed: Jan. 17, 2006

(51) Int. Cl. A61M 25/00

(52) U.S. Cl. 600/585

(57) ABSTRACT

Occluding guidewires to access bodily lumen of a patient are disclosed. The occluding guidewires include a slotted tube and a balloon secured to a distal end of the slotted tube. The slotted tube includes a plurality of slots. The slots are in fluid communication with a lumen defined by the slotted tube. The balloon is secured about the slotted tube over one or more slots to permit inflation media to be communicated through one or more slots from the lumen into the inflation chamber of the balloon. The occluding guidewire may also include a proximal tube. A proximal lumen of the proximal tube is in communication with the lumen of the slotted tube. The occluding guidewire may be configured with a relatively small diameter using aspects of the present inventions.

### BACKGROUND OF THE INVENTION

1. Summary of the Invention

The present inventions relate to medical devices and, more particularly, to low-profile balloon catheters and occluding guidewires for introduction of fluids into patients.

2. Description of the Related Art

Medical catheters and guidewires can be useful tools in treating intravascular disorders, disorders within other lumen of the body, extracting fluids from lumen as well as introducing fluid into lumen. Some medical devices are configured to receive a medical catheter or guidewire to permit the medical device to be positioned within the body of a patient. Most medical devices are configured to receive a medical catheter or guidewire having an outside diameter of around 0.014 inches. Further, many catheters and some guidewire designs can permit the inclusion of a balloon at or near the distal end of the catheter or guidewire. Depending on the configuration, these devices can also be used to introduce and/or expand various other medical devices, such as stents for example. A balloon may help to direct the distal end of the catheter through a lumen where the pulsatile flow of blood may the balloon to act as a “sail.” Further, the balloons in various configurations may be used to test for flow paths using occlusion of vessels, for embolization of vasculature, to treat or control vasospasms, and for treatment of nosebleeds, among other uses. In particular, silicone balloons have been shown to be effective for treating vasospasms.

Further, there are significant benefits in reducing the diameter of medical catheters and guidewires for many applications. The reduced size is generally less traumatic to a patient. The reduced size also permits access to locations of reduced size or diameter that may not be reachable or treatable by larger diameter medical catheters and guidewires. Various locations in the brain or heart can be particularly difficult to reach and/or treat when the area to be treated or otherwise accessed is within a vessel having a relatively small diameter. This is, at least in part, due to the tortuous path that must be navigated to access some locations as well as the point for introduction of the medical catheter or guidewire frequently being the femoral artery. Reduced diameter medical catheters and guidewires may have physical characteristics that may enhance the difficulty of placing them at such remote locations. Accordingly, a need exists for components that provide performance characteristics that simplify the placement of reduced diameter medical catheters and guidewires.

Manufacturing medical devices can be difficult. As the size of the devices decreases, the difficulty in manufacturing the devices generally increases. Medical catheters and guidewires have outside diameters as small as about 0.010 inches. Accordingly, a need exists for configurations of components that provide for simplified manufacture of reduced diameter medical catheters and guidewires.

### SUMMARY OF THE INVENTION

Apparatus and methods in accordance with the present inventions may resolve many of the needs and shortcomings discussed above and will provide additional improvements and advantages as will be recognized by those skilled in the art upon review of the present disclosure.

The present inventions provide an occluding guidewire in the form of an elongated hollow tube having a distal balloon secured to its distal end. The occluding guidewire in accordance with the present invention may be formed from one or more tubes defining a continuous lumen. The tubes may be formed by extrusion and drawing and typically have a sufficiently stiff proximal end, a flexible, atraumatic distal end, and a wall thickness that optimizes the cross-sectional area of the lumen for particular applications. In one aspect, small bodily lumens of a patient can be accessed with an occluding guidewire in accordance with the present invention by conventional guidewire techniques. Small bodily lumens can be accessed by the occluding guidewire and occluded using the distal balloon to provide certain types of diagnosis and/or treatment at the desired location within the body while also enabling access of conventional over-the-wire instruments. The small outer diameter of the occluding guidewire can enable conventional over-the-wire instruments to be used in conjunction with the occluding guidewire.

In one aspect, the occluding guidewire can be constructed for insertion into the body to occlude blood flow in an artery. The occluding guidewire having a proximal tube extending a proximal portion of the length of the occluding guidewire, and a slotted tube secured to the proximal tube. The slotted tube includes a distal end and a flexible section having a plurality of slots cut into the slotted tube. The plurality of slots can be formed perpendicular to the longitudinal axis of the slotted tube.

The plurality of slots can also be formed at oblique angles to the longitudinal axis of the slotted tube. The size, shape and pattern of the slots may be altered to provide the desired flexibility to the slotted tube for particular applications of the occluding guidewire. The plurality of slots may all be formed at the same angle or the varying angles along a length of the slotted tube. The plurality of slots can alternately be formed on opposite sides of the slotted tube, may be formed on one side or may be formed at varying locations about the circumference of the slotted tube. The plurality of slots may be formed to generally increase the flexibility of the slotted tube or may be formed to provide increased flexibility in one or more directions. The width of the slots may vary along the length of the slotted tube such that the width is less in proximal portions of the slotted tube, e.g., from about 0.001 inches to about 0.002 inches, relative to distal portions thereof, e.g., from about 0.004 inches to about 0.005 inches. Alternatively, the width of the slot may be constant, e.g., between about 0.002 inches to about 0.004 inches. In certain embodiments, the slots may extend over a distal section of the slotted tube over about the last 3 to 5 cm of the length of the slotted tube.

In one aspect, the proximal tube and/or the slotted tube can be formed from stainless steel. In another aspect, the proximal tube and/or the slotted tube can be formed from nitinol. One or both of the proximal tube and the slotted tube may be annealed progressively to vary the flexibility along the length of the proximal and the slotted tubes. The distal portion of the proximal tube may be annealed such that the distal portion has greater flexible than proximal portions of the slotted tube.

When the occluding guidewire includes a proximal tube and a slotted tube, the distal end of the proximal tube is secured to the proximal end of the slotted tube. In one aspect, a distal notch may be formed at the distal end of the proximal tube and a proximal notch may be formed at the proximal end of the slotted tube. The notches may be integrally formed with the tubes, may be formed by cutting or grinding, or may be otherwise formed. The proximal notch and the distal notch can be overlapped and secured to one another using adhesives, welding or other techniques.

An inner tube or lumen liner may extend along at least a portion of the slotted tube. The inner tube may be secured to the wall of the slotted tube at its distal end and may be secured to the wall at locations both proximal and distal to the plurality of slots not covered by a balloon. The lumen liner extends over at least the plurality of slots not covered by a balloon. Both the inner tube and the lumen liner may permit the passage of an inflation media through the lumen of the slotted tube.

An atraumatic tip secured to the distal end of the slotted tube may have a rounded tip for atraumatic insertion into the body. The atraumatic tip may be fabricated from a metal or may be a polymeric material such as PET, polyimide, or polyethylene. The atraumatic tip may include a shaping wire secured to or within the distal end of the slotted tube. A coil may extend around the shaping wire. The rounded tip may be secured to one or both of the coil and the shaping wire. The atraumatic tip may include one more components containing a radiopaque material.

In another aspect, the present inventions feature methods of treating a patient using the occluding guidewire. The occluding guidewire is inserted into a body lumen and guided to a target location in the lumen requiring treatment. Once positioned at the target location, the balloon may be inflated. Inflation media is passed through the occluding guidewire and exits the occluding guidewire though one or more of the plurality of slots in the slotted tube to enter the balloon or, in some embodiments, a bodily lumen at the desired location. Further, a surgical instrument may be slid, in guided contact, over the occluding guidewire to access the desired location such that at least one surgical operation using the surgical instrument may be performed at the desired location. After the treatment, the distal balloon is typically deflated by withdrawing inflation media through one or more of the plurality of slots. The occluding guidewire is removed from the body lumen.

Other features and advantages of the invention will become apparent from the following detailed description, and from the claims.

### BRIEF DESCRIPTION OF THE DRAWINGS

FIG. 1 illustrates a perspective view of an embodiment of an occluding guidewire in accordance with the present inventions;

FIG. 2A illustrates a cross section of a partial side view of an embodiment of an interconnection between a proximal tube and a slotted tube in accordance with the present inventions;

FIG. 2B illustrates a cross section of a partial side view of another embodiment of an interconnection between a proximal tube and a slotted tube in accordance with the present inventions showing the intersection between the two tubes in phantom;

FIG. 2C illustrates a cross section of a partial side view of another embodiment of an interconnection between a proximal tube and a slotted tube in accordance with the present inventions showing the intersection between the two tubes in phantom;

FIG. 3A illustrates a partial side view of a length of the slotted tube showing an exemplary embodiment for the slots;

FIG. 3B illustrates a partial side view of a length of the slotted tube showing another exemplary embodiment for the slots;

FIG. 3C illustrates a partial side view of a length of the slotted tube showing annother exemplary embodiment for the slots;

FIG. 4 illustrates a perspective view of the distal end portion of an exemplary embodiment of an apparatus in accordance with the present inventions

FIG. 5A illustrates a cross section of partial side view at the distal end of the slotted tube showing an exemplary embodiment of slots in the slotted tube positioned under the balloon;

FIG. 5B illustrates a cross section of partial side view at the distal end of the slotted tube showing an exemplary embodiment of a sealant extending proximally from the balloon;

FIG. 5C illustrates a cross section of partial side view at the distal end of the slotted tube showing an exemplary embodiment of a fluid tube extending proximally from the balloon; and

FIG. 6 illustrates a cross-section of a partial side view at the distal end of the slotted tube showing an exemplary embodiment of the balloon in at least a partially inflated with an inflation media.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements for various applications will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with an apparatus in accordance with the present invention.

### DETAILED DESCRIPTION OF THE INVENTION

The figures generally illustrate embodiments of occluding guidewires 10 including aspects of the present inventions. The particular exemplary embodiments of the occluding guidewires 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, many variations from the illustrated embodiments may be encompassed by the appended claims.

The present inventions provide occluding guidewires 10 and associated methods. Occluding guidewires 10 in accordance with the present inventions may permit the passage of fluid through a lumen to or from a balloon 16 while maintaining an enhanced flexibility at the distal end of the occluding guidewire. The fluids typically introduced into the distal balloon 16 may include various types of inflation media. Inflation media frequently include various imaging compounds and may include various medicinal or other compounds that can be desirable in particular applications.

As generally illustrated throughout the Figures, occluding guidewires 10 generally include a proximal tube 12, a slotted tube 14 and a balloon 16. In some aspects, the occluding catheter 10 may only include a slotted tube 14 and a balloon 16. In still other aspects, the slotted tube 14 may not include a balloon 16. The slotted tube 14 defines a plurality of slots 18 extending between an outer surface 64 of the slotted tube 14 and an inner surface of the slotted tube which defines a lumen 24. The balloon 16 alone or in combination with the slotted tube 14 defines an inflation chamber 26 to a fluid to inflate the balloon 16. The balloon 16 is secured about the distal end or distal portion of the slotted tube 14 with the inflation chamber 26 in fluid communication with at least one of the slots 18. For inflation, the balloon 16 receives fluid from the lumen 24 through the one or more slots 18 in communication with the inflation chamber 26. For deflation, the lumen 24 receives fluid from the balloon 16 through the one or more slots 18 in communication with the inflation chamber 26. When included, the proximal tube 12 defines a proximal lumen 22 that is in fluid communication with the lumen 24 of the slotted tube 14 at a proximal end 114 of the slotted tube 14. Various connectors may be provided at the proximal end 112 of the proximal tube 12 or elsewhere along the proximal tube 12 depending upon the configuration of the occluding catheter 10 to assist in communicating fluids into the proximal lumen 22. When a proximal tube 12 is not included in the occluding guidewire 10, various connectors may be provided at the proximal end 114 of the slotted tube 14 or elsewhere along the slotted tube 14 depending upon the configuration of the occluding catheter 10 to assist in communicating fluids into the proximal lumen 22. The connectors may include Leur lock port, a Touhy Borst port, side arm port or other port or fitting through which the connector may receive the fluid. Further, a handle may be secured to the proximal tube 12 or slotted tube 14 to assist in the manipulation of the catheter 10 within a patient. For purposes of this description, occluding guidewires 10 generally define a longitudinal axis 300 extending along their length as illustrated in the Figures.

The proximal lumen 22 of the proximal tube 12 extends over at least a portion of the length of the proximal tube 12. The proximal lumen 22 may extend between a first proximal tube opening 32 and a second proximal tube opening 42 defined by the proximal tube 12. The proximal tube 12 may further define a proximal notch 52 at a second end 212 of the proximal tube 12. The proximal notch 52 generally extends from the second end 212 of the proximal tube 12 to a location along the tube which is proximal to the second end 212 of the proximal tube 12. The proximal notch 52 is generally shaped to receive a distal notch 54 at a first end 214 of slotted tube 14. The proximal notch 52 may extend into the proximal lumen 22. The proximal lumen 22 and the lumen 24 together may form a continuous passage extending between a first end 112 of proximal tube 12 and a slot 18 in slotted tube 14. When present, the proximal tube 12 is generally configured to guide and position portions of the slotted tube 14 within a patient.

The proximal lumen 22 of the proximal tube 12 generally extends longitudinally within the proximal tube 12 from a first proximal tube opening 32 to a second proximal tube opening 42 defined by the proximal tube 12. The proximal lumen 22 is generally configured to receive a fluid, such as for example inflation media, and communicate the fluid at least to the second proximal tube opening 32.

The proximal tube 12 may be configured to have a desired balance of longitudinal stiffness and torsional rigidity based on the characteristics of the slotted tube 14. The longitudinal stiffness, at least in part, dictates the push characteristics for the proximal tube 12. The torsional rigidity, at least in part, dictates the precision of the rotational control provided by the proximal tube 12. The proximal tube 12 may be made from a variety of materials including polymers, metals, and various composite materials. In one aspect, the proximal tube 12 is made of a stainless steel. In another aspect, the proximal tube 12 is made of nitinol. Typically, the proximal tube 12 is configured to have a desired elastic range.

The proximal tube 12 may have various diameters and lengths depending on the particular application for the occluding catheter 10. Generally, the proximal tube 12 is configured to at least support inflation of the associated balloon 16. The particular configuration of proximal tube 12 may also depend upon whether or not the proximal tube 12 is intended primarily for use as a balloon catheter or as a wire support for other guidewires or catheters. For use primarily as a balloon catheter, an occluding catheter 10 may be configured to support larger volumes of fluid than when the occluding catheter 10 is configured for use as a delivery rail for other medical devices. In such applications for balloon inflation, the tube may have an outside diameter of about 0.024 inches and a lumen diameter of about 0.019 inches. This outside diameter can provide the desired torsional rigidity without being too longitudinally stiff. The diameter of the proximal lumen 22 may be selected to provide a desire inflation/deflation time. For intercranial applications where the insertion point is in the femoral artery, the length of the proximal tube 12 may be about 110 centimeters. A proximal tube 12 of this length may keep the proximal tube 12 in the straight portion of the guide. For use of occluding catheter 10 as a guide wire, the outside diameter may be around 0.014 inches. For other applications requiring access to smaller bodily lumen, an outside diameter of less than 0.014 inches may be used. In one exemplary embodiment, the proximal tube 12 of the occluding catheter 10 can have an outer diameter 312 of the order of 0.014 inches and a wall thickness 412 of the order of 0.002 inches to maximize the diameter 322 of the proximal lumen 22. The proximal tube can be between about 165 cm to about 205 cm in length and although flexible, have a stiffness of about 50-100 N-mm² to impart sufficient lateral stiffness and torque transmission capabilities along its length.

The slotted tube 14 defines a lumen 24 extending along at least a portion of the length of the slotted tube 14. The lumen 24 is in fluid communication with a first tube opening 34 and one of the plurality of slots 18. The lumen 24 may extend between a first tube opening 34 and a second tube opening 44 defined by the slotted tube 14. When occluding catheter 10 includes a proximal tube 12, the first end 114 of the slotted tube 14 is typically secured to a second end 212 of the proximal tube 12. The slotted tube 14 is secured to the proximal tube 12. The proximal tube 12 is typically secured to the slotted tube 14 such that a proximal lumen 22 of the proximal tube is in fluid communication with the lumen 24 of the slotted tube 14. For purposes of the present inventions, the term “secured to” means that proximal tube 12 is attached to the slotted tube 14 by a suitable method such as, for example, by welding, brazing, heat shrinking, or gluing among other methods. To secure the slotted tube 12 to the proximal tube, the slotted tube may define a distal notch 54 to receive a proximal notch 52 of the proximal tube 12.

The proximal tube 12 and the slotted tube 14 may be secured to one another in an end to end, abutting relationship or in an overlapping relationship. The slotted tube 14 generally directs a fluid from a first end 114 of slotted tube 14 to a second end 214 of the slotted tube 14 or to a location adjacent to the second end 214 of slotted tube 14. The lumen 24 is defined by the inner surface 64 of the slotted tube 14. The lumen 24 generally extends longitudinally within the slotted tube 14 from a first slotted tube opening 36 to a second slotted tube opening 46 defined by the slotted tube 14. The slotted tube 14 is also generally configured to permit the distal end 114 of slotted tube 14 to be positioned at a desired location within a bodily lumen of a patient. The slotted tube 14 may be made from a range of materials that will be recognized by those skilled in the art as depending upon the intended use for the resultant occluding catheter 10. In one aspect, the slotted tube 14 can be made from one or more polymers such as polyethylene, nylon, and polyimide, for example, among others. In other aspects, the slotted tube 14 can be made from one or more metals such as stainless steel or nitinol, for example, among others. The slotted tube 14 defines at least one lumen 24 to permit the communication of fluids along at least a portion of the length of the slotted tube 14. The slotted tube 14 typically has an outside diameter 314 which is the same or smaller than the outside diameter 312 of the proximal tube 12. For use primarily as a balloon catheter, the slotted tube 14 may have an outside diameter 314 of 0.024 inches. The proximal lumen 26 may then have a diameter 322 of about 0.020 inches. The lumen 24 may be configured with as large a cross sectional area as possible given the size and application for the occluding catheter 10. In one exemplary embodiment, the slotted tube 14 of an occluding catheter 10 has a length from about 15 cm to about 25 cm. The slotted tube has an outside diameter 314 of about 0.014 inches and is secured to a proximal tube 12 having the same outside diameter 312. Slotted tube 14 may have a stiffness of about 25-50 N-mm² or less, to impart the desired flexibility to occluding catheter 10. Additionally, the flexibility of occluding catheter 10 may be varied by progressively annealing either a portion of the occluding catheter 10, for example, only slotted tube 14, or progressively annealing the entire length of occluding catheter 10.

The plurality of slots 18 extend from an outer surface 64 of the slotted tube 14 to the inner surface 64 defining lumen 24. The slots 18 may be formed in the slotted tube 14 by various techniques such as, for example, EDM machining, chemical masking, electrochemical etching, or laser etching among others. The slots 18 may function to modify the flexibility of the slotted portions of the slotted tube 14 and may also allow the communication of fluid from the lumen 24 into the inflation chamber of a balloon 16. The slots 18 generally extend diametrically across the slotted tube 14. In some aspects, the slots 18 may be perpendicular to a longitudinal axis 300 of the occluding catheter 10 and in other aspect, the slots may be at oblique or obtuse angles to the longitudinal axis. Typically, the slots 18 are substantially planar. The width of the slots 18 may vary along the length of the slotted tube 14 in one aspect the slots 18 may vary progressively along the length of the slotted tube 14 such that the width is less in proximal portions of the slotted tube 14 such as for example from about 0.001 inches to about 0.002 inches, relative to distal portions thereof such as for example from about 0.004 inches to about 0.005 inches. Alternatively, the width of the slots 18 may be constant such as for example between about 0.001 inches to about 0.008 inches.

A balloon 16 is positioned over one or more slots 18 which are in fluid communication with the lumen 24. Typically, balloon 16 is positioned at or near the second end 214 of the slotted tube 14. The balloon 16 may define an inflation chamber 30. The inflation chamber 30 is in fluid communication with the lumen 24 by one of a plurality of slots 18 extending between the outer surface 64 of the slotted tube 14 and the lumen 24 to permit inflation media to be introduced or removed from the inflation chamber 30. In another aspect, the second tube opening 44 may also be in fluid communication with the inflation chamber 30.

A balloon 16 may be provided at or near the second end 216 of the slotted tube 14 tube. The balloon 16 may be configured as a balloon to be received over the distal end of the slotted tube 14 or may be configured as a tube sealingly secured to the slotted tube 14 at proximal and distal locations along the length of the slotted tube 14. Regardless of configuration, the balloon 16 defines an inflation chamber 30. The inflation chamber 30 of the balloon 16 is in fluid communication with at least one of the slots 18 of the slotted tube 14 to allow the communication of inflation media to and from the inflation chamber 30 of balloon 16. Depending upon the application for the occluding catheter 10, the balloon 16 may be either compliant or non-compliant. For vascular applications, the balloon form or tube of balloon 16 may be configured and sized to provide the correct inflated diameter and length for target vessel treatment locations. Target vessel diameters can range from as large as 10 to 12 millimeters to as small as 2 to 3 millimeters with many different lengths needed. In compliant embodiments, the balloon 16 may be made from silicone. For neurovascular applications, silicone may provide additional therapeutic benefits relating to spasms that will be recognized by those skilled in the art upon review of the present disclosure. When silicone is used, the silicone material may have a durometer of about 20 to 30. For neurovascular applications, this may give the occluding catheter 10 the correct ‘feel’ when the balloon 16 is inflated to a pressure of about 1 atmosphere.

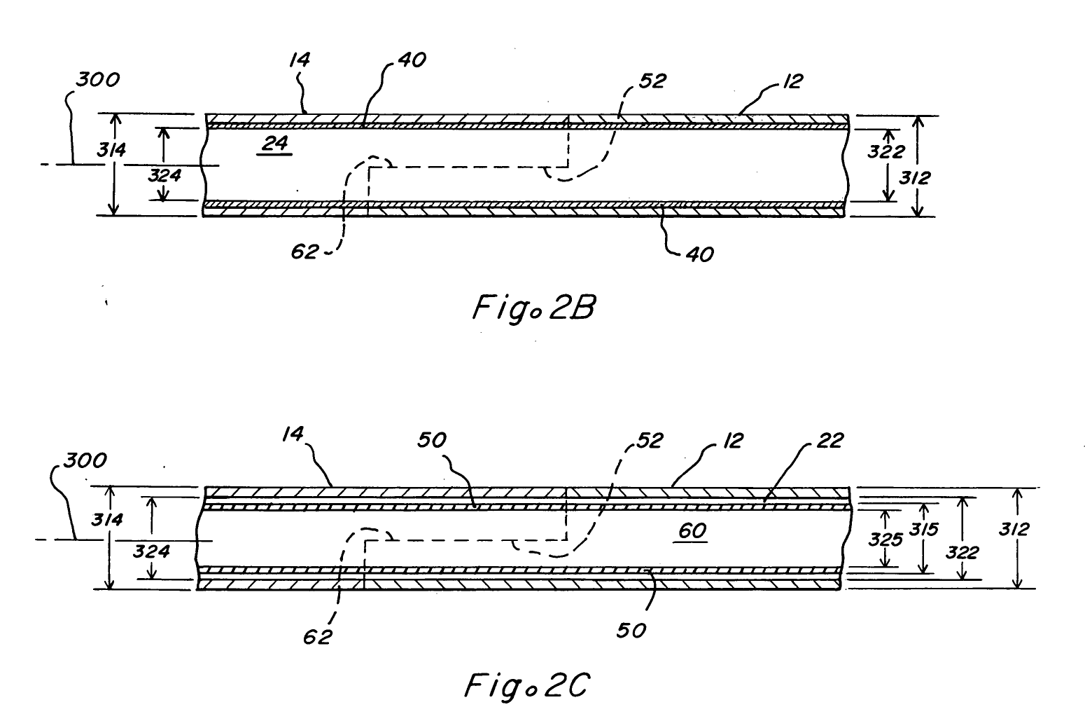

A lumen liner 40 may be provided along the lumen 24 of the slotted tube 14. The lumen liner 40 may seal slots 18 positioned proximal to the proximal end of the balloon 16 while allowing fluid to be communicated along lumen 24. The lumen liner 40 may also modify the torsional rigidity and longitudinal stiffness of the slotted tube 14. The lumen liner 40, shown in FIGS. 2B, 3A and 4B, may be secured to, biased against or deposited on the inner surface 64 defining the lumen 24 of the slotted tube 14. The lumen liner 40 typically extends from at least the first slot 18 proximal to the proximal end of the balloon 16 to the most proximal slot 18 along the length of the slotted tube 14. In one aspect, lumen liner 40 is a coating on the inner surface 64 of the slotted tube 14 defines the lumen 24 of the slotted tube 14. The lumen liner 40 may formed to maximize the inside diameter 324 of the slotted tube 14 and, thus, the diameter of lumen 24, while adequately sealing the slots 18 positioned proximal to the proximal end of balloon 16. Alternatively, lumen liner 40 may be a sleeve that is radially expanded from inside lumen 24 of slotted tube 14. In such an embodiment, the lumen liner 40 is expanded sufficiently to contact the inner surface 64 of slotted tube 14 which defines lumen 24.

An inner tube 50, shown in FIGS. 2C, 3B, 3C and 5C, may be secured within the lumen 24 of the slotted tube 14. The inner tube 50 may seal and/or provide additional strength to slotted tube 14. The inner tube 50 includes an inflation lumen 60 to permit inflation of the balloon 16 when slotted tube 14 includes slots 18 positioned proximal to the proximal end of the balloon 16. Typically, inner tube 50 is sealingly secured to the inner surface 64 of slotted tube 14 at least at a distal end of the inner tube 50. The inner tube 50 may otherwise be slidable relative to the slotted tube 14. If the inner tube 50 is secured along a substantial portion of the length of the slotted tube 14 or at multiple locations along the length of the slotted tube 14, the inner tube 50 may modify the torsional rigidity and longitudinal stiffness of the slotted tube 18. When the inner tube 50 is sealingly secured to the inner surface 64 of the slotted tube 14 at both its proximal and distal end, the inner tube 50 typically extends from at least the first slot 18 proximal to the proximal end of the balloon 16 to the most proximal slot 18 along the length of the slotted tube 14. Alternatively, inner tube 50 may be a sleeve that is radially expanded from inside the lumen 24, to contact the inner surface 64 of slotted tube 14 defining lumen 24 and seal slots 18 along its length. In other aspects, the inner tube 50 may extend to the proximal end of the slotted tube 14 and, in embodiments with a proximal tube, to the proximal end of the proximal tube 12 through the proximal lumen 22. The inner tube 50 may be fabricated from metals such as for example, stainless steel and nitinol or from polymers such as for example PET, polyimide, polyethylene, polyurethane, teflon, EVA, silicone, or hydrophilic gel.

An atraumatic tip 90 may be attached to the distal end 114 of the slotted tube 14. The atraumatic tip 90 may include a coil 96 such as, for example, a spring coil. The coil 96 may be about 2 cm long and about 0.014 inches in diameter. The coil 96 can be made of 0.002 inches in diameter radio opaque material, preferably platinum. However, other materials known in the art can be used for the coil 96 as well. The atraumatic tip 90 generally provides a soft, gentle bumper for the distal end 114 of the slotted tube 14. A shaping ribbon 98 may be positioned within the coil 96. The shaping ribbon 98 is typically constructed from a metal and can serve several important functions. The shaping ribbon 98 may serve as a bendable beam to more easily permit a user to induce a curved shape in the atraumatic tip 90 to direct the occluding catheter 10 through a bodily lumen of a patient. Further, the shaping ribbon 98, may improve the safety of an occluding catheter 10 by not allowing the coils 96 of the atraumatic tip 90 to stretch out if a portion of the atraumatic tip 90 becomes lodged or otherwise hung up in the bodily lumen of a patient. The first end of the shaping ribbon 98 may be attached to the second end 114 of the slotted tube 14 and/or the proximal ends of the coils 96 and the second end is secured to the distal end of the coils 96. The size of the shaping ribbon 98 may be about 0.002 inches by 0.004 inches. The shaping ribbon 98 is made from a material having the desired combination of ductility and elasticity. Stainless steel of a proper temper is commonly used to provide these characteristics. The coil 96 may terminate in a round spherical shape cap so it is generally atraumatic to the wall of a bodily lumen.

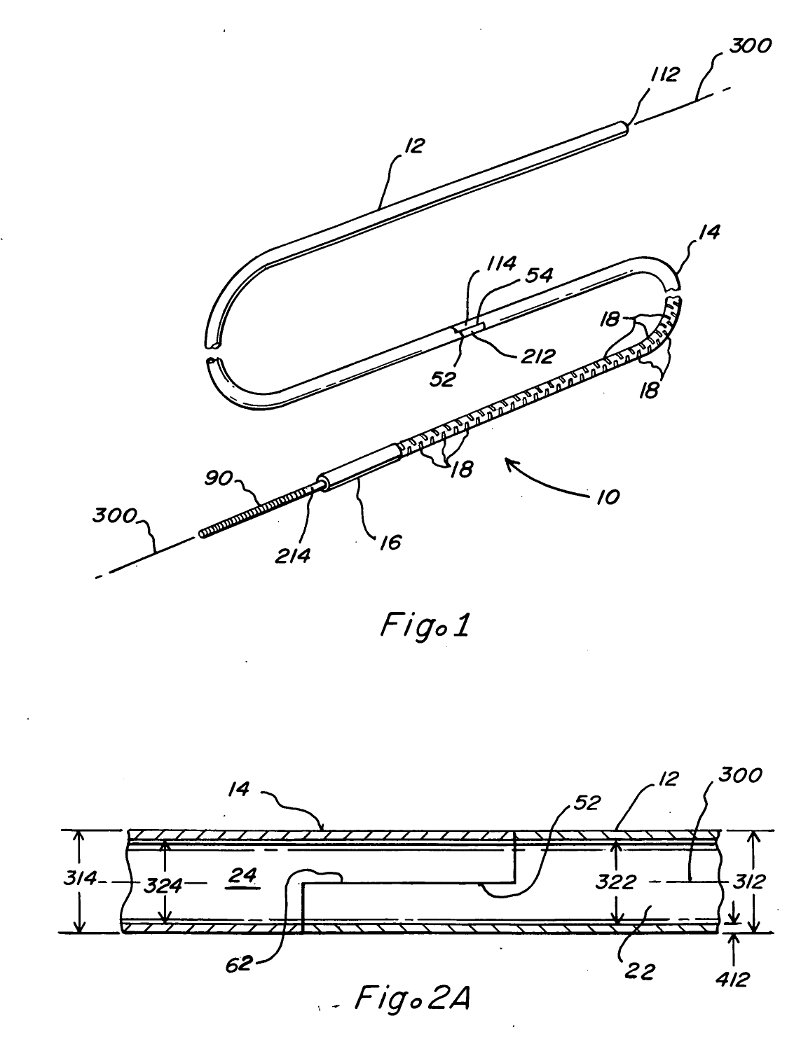

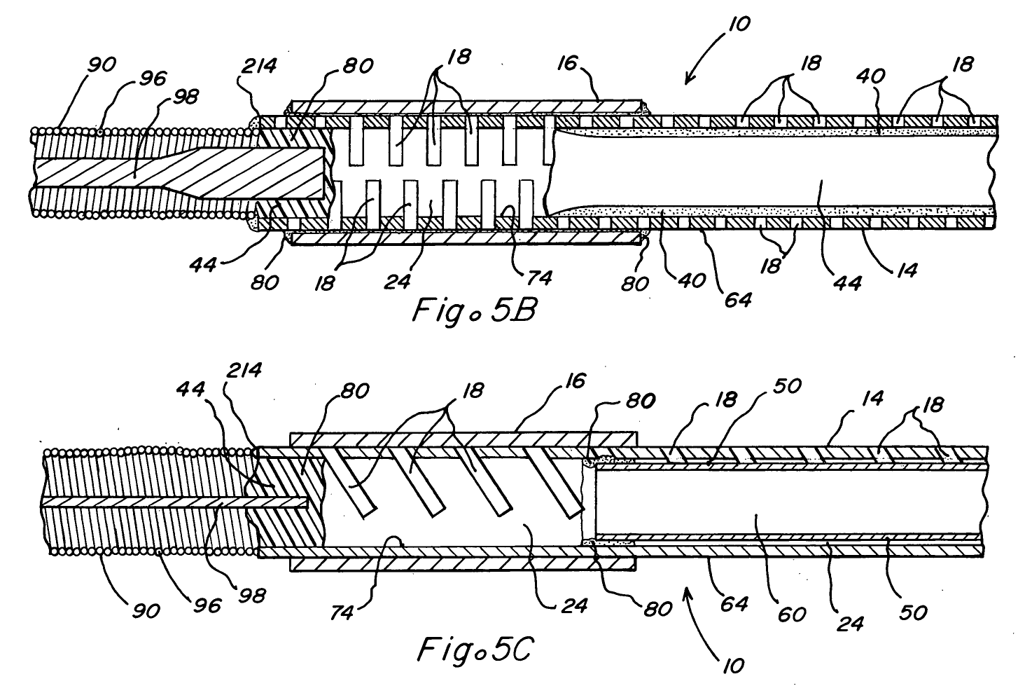

FIG. 1 illustrates an exemplary embodiment of an occluding catheter 10 in accordance with the present inventions including both a proximal tube 12 and a slotted tube 14. The proximal tube and slotted tube are illustrated as having a circular cross section for exemplary purposes. The illustrated embodiment includes a passage extending from a first end 112 of the proximal tube 12 to one or more slots 18 underlying the balloon 16 at a region proximal to the second end 214 of the slotted tube 14 to communicate inflation media from the first end 112 of the proximal tube 12 to the inflation chamber 30 of the balloon 16. The passage is formed by connecting the proximal tube 12 to the slotted tube 14 such that the proximal lumen 22 of the proximal tube 12 is in fluid communication with the lumen 24 of the slotted tube 14. As illustrated, the proximal tube 12 is secured to the slotted tube by overlapping a proximal notch 52 in the proximal tube with a distal notch 54 in the slotted tube 14. The slotted tube 14 is illustrated with a plurality of slots 18 positioned proximal to the proximal end of balloon 16. To permit inflation, a lumen liner 40, shown in FIGS. 2B and 5B, or an inner tube 50, shown in FIGS. 2C and 5C, may be provided within lumen 24 to prevent the passage of fluid through at least the slots 18 not covered by the balloon 16. An atraumatic tip 90 is shown secured to the second end 214 of slotted tube 14. In the illustrated embodiment, the slotted tube 14 is generally configured to be directed through a bodily lumen within a patient by a physician manipulating the proximal tube 12 and to inflate the balloon 16 for diagnostic or therapeutic purposes.

FIGS. 2A to 2C illustrate a cross-section through the longitudinal axis at the intersection of the proximal tube 12 and the distal tube 14 of exemplary embodiments of apparatus in accordance with the present invention. As illustrated for exemplary purposes, the proximal tubes and distal tubes have the same outside diameters 312, 314 and the same inside diameters 322, 324. In certain applications varying outside diameters 312, 314 and inside diameters 322, 324 may be utilized to meet particular performance requirements.

FIG. 2A illustrates embodiments of a proximal tube 12 having a proximal notch 52 and a distal tube 14 having a distal notch 62. The proximal notch 52 of the proximal tube 12 is overlapped with the distal notch 62 of the distal tube 14. The surfaces defining the proximal notch 52 and distal notch 62 are then secured to one another to interconnect the proximal tube 12 and the distal tube 14. The surfaces of the notches 52, 62 may be welded, adhesively bonded, or otherwise secured to one another.

FIG. 2B illustrates embodiments of a proximal tube 12 and a slotted tube 14 secured together at notches 52, 62 similar to the embodiment of FIG. 2A. However, the embodiment of FIG. 2B includes a lumen liner 40 secured to the inner surface 72 of the proximal tube 12 and to the inner surface 74 of slotted tube 14. The lumen liner 40 as illustrated extends over the junction of the proximal notch 52 and distal notch 62 and, accordingly, the junction is illustrated in phantom. The lumen liner 40 is secured peripherally about the proximal lumen 22 and/or lumen 24 and is configured to retain an inside diameter 322, 324 or cross-sectional area in an otherwise shaped proximal lumen 22 and/or lumen 24 to permit an adequate flow of inflation media through the proximal lumen 22 and/or lumen 24. In one aspect, the lumen liner 40 may seal any gaps in the junction between the proximal notch 52 and distal notch 62. Alternatively or additionally, the lumen liner 40 may be configured to modify the physical and/or performance characteristics of the proximal tube 12, the slotted tube 14 and/or the junction between the proximal tube 12 and the slotted tube 14.

FIG. 2C also illustrates embodiments of a proximal tube 12 and a slotted tube 14 secured together at notches 52, 62 similar to the embodiments of FIGS. 2A and 2B. However, the embodiment of FIG. 2C includes an inner tube 50 positioned within and extending between the proximal lumen 22 of the proximal tube 12 and the lumen 24 of the slotted tube 14. The inner tube 50 defines an inflation lumen 60. The inflation lumen 60 may be configured to permit the flow of inflation media to and from balloon 16. In this aspect, the inflation lumen 60 typically has an inside diameter 325 to permit an adequate flow of inflation media through the inflation lumen to meet the performance requirements for the occluding catheter 10. The inner tube 50 may be secured to the inner surface 72 of the proximal tube 12 and to the inner surface 74 of slotted tube 14 at one or more points along their length. As illustrated, the inner tube 50 has an outer diameter 315 less than the inner diameters 322, 324 of the proximal lumen 22 and lumen 24. The inner tube 50 extends over the junction of the proximal notch 52 and distal notch 62 and, accordingly, the junction is illustrated in phantom. At the illustrated location, the inner tube 50 may eliminate the need to seal any gaps in the junction between the proximal notch 52 and distal notch 62 as inflation media may be communicated through the inflation lumen 60. Alternatively or additionally, the inner tube 50 may be configured to modify the physical and/or performance characteristics of the proximal tube 12, the 25 slotted tube 14 and/or the junction between the proximal tube 12 and the slotted tube 14.

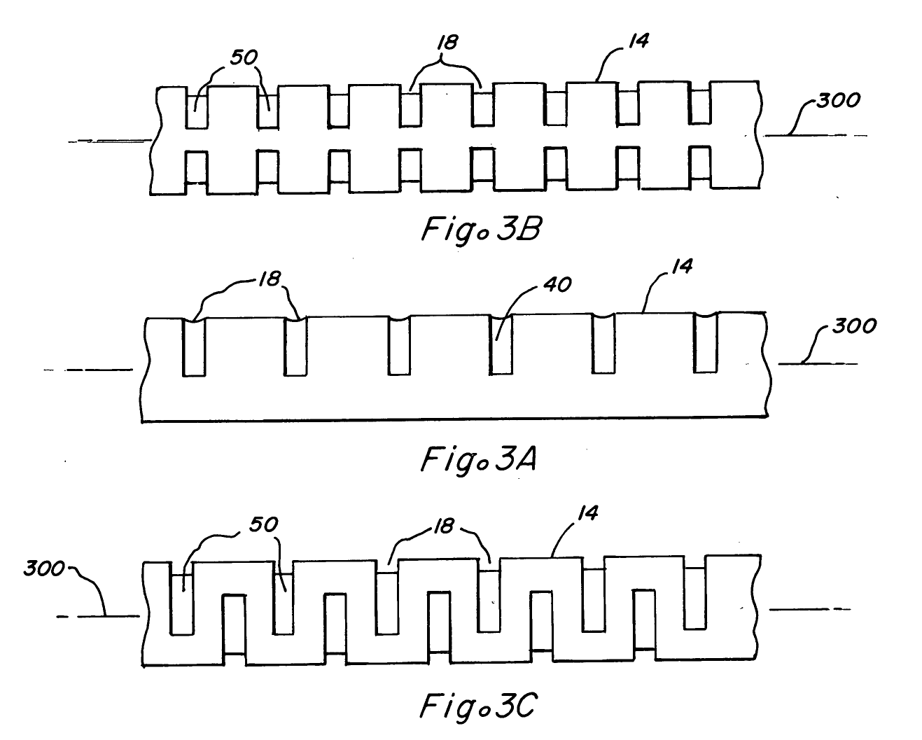

FIGS. 3A to 3C illustrate exemplary embodiments for slots 18 in a slotted tube 14. FIG. 3A illustrates an exemplary pattern of slots 18 along a single side of slotted tube 14. Further, a lumen liner 40 is illustrated as extending into slots 18 for exemplary purposes. The slots 18 extend through the slotted tube 14 to about the longitudinal axis 300 of the slotted tube 14. In this pattern, the slots 18 may enhance the flexibility of the slotted tube 14 uni-directionally. FIG. 3B illustrates an exemplary pattern of slots 18 along opposing sides of a slotted tube 14. Further, an inner tube 50 is illustrated as extending between one or more slots 18 for exemplary purposes. The slots 18 on the opposing sides are equally spaced and extend through the slotted tube 14 in an opposing configuration. Accordingly, the slots 18 extend through the slotted tube 14 up to a desired distance from the longitudinal axis 300 of the slotted tube 14. In this pattern, the slots 18 may enhance the flexibility of the slotted tube 14 bi-directionally. FIG. 3C illustrates another exemplary pattern of slots 18 along opposing sides of a slotted tube 14. Again, an inner tube 50 is illustrated as extending between one or more slots 18 for exemplary purposes. The slots 18 on the opposing sides are equally spaced and extend through the slotted tube in an alternating configuration. As illustrated, the slots extend through the slotted tube 14 through the longitudinal axis 300 of the slotted tube 14. In this pattern, the slots 18 may enhance the flexibility of the slotted tube 14 omni-directionally. Additional enhancements to flexibility may be achieved using alternative slot patterns as will be recognized by those skilled in the art upon review of the present disclosure.

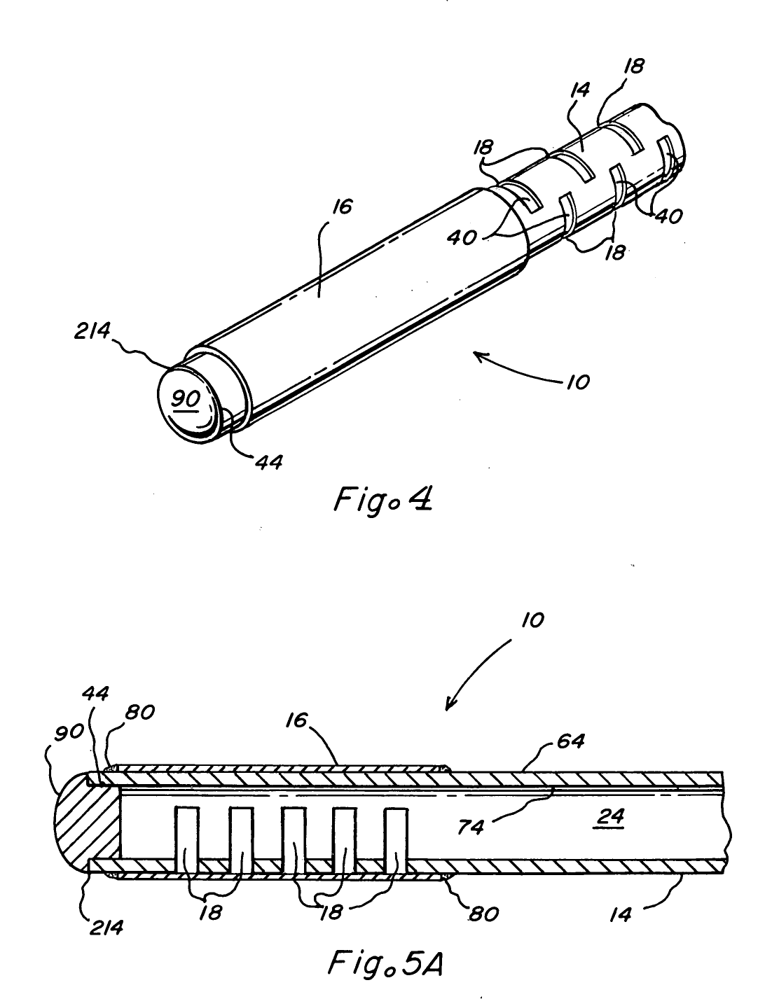

FIGS. 4 to 6 illustrate exemplary embodiments for a distal portion of the slotted tube 14. FIG. 4 illustrates a detailed perspective view of the distal portion of a slotted tube 14 including a balloon 16. As illustrated, the balloon 16 is in the form of a tube secured over the slotted tube 14. The balloon 16 is secured with the distal end 216 of the balloon 16 positioned proximal to the distal end 214 of the slotted tube 14. When configured as a tube, the balloon 16 is typically secured to the slotted tube 14 at the proximal end 116 and the distal end 216 of the tube with the distal end 216 of the tube positioned at a location at or proximal to the distal end 214 of the slotted tube 14. This permits the expansion of the balloon 16 at locations intermediate to the proximal end 116 and distal end 216 of the balloon 16 as inflation media is communicated through slots 18 into the inflation chamber 30 of the balloon 16. More generally, the proximal end 116 of the balloon 16 is secured proximal to the distal end 214 of the slotted tube 14 such that at least one slot 18 is in fluid communication with the expansion chamber 30 of the balloon 16. The slots 18 proximal to the proximal end 116 of the balloon 16 are illustrated as sealed with a lumen liner 40 for exemplary purposes. An atraumatic tip 90 is provided at the distal end 214 of the slotted tube 14 and, as illustrated, may function to seal the second slotted tube opening 44 of the slotted tube 14.

FIG. 5A illustrates a cross sectional view of another exemplary embodiment of the distal portion of a slotted tube 14 with the balloon 16 in an unexpanded position. As illustrated, the slotted tube 14 does not include any slots 18 positioned proximal to the proximal end 116 of balloon 16. The slots 18 extend along the slotted tube 14 between a proximal end 116 and a distal end 216 of the balloon 16. The slots 18 are in fluid communication with a surface of the balloon 16 which form expansion chamber 30 or may be expanded to form an expansion chamber 30 upon the introduction of an inflation media. An adhesive 80 is provided at the proximal end and the distal end of the balloon 16 to secure the proximal end and the distal end of the balloon 16 to the outer surface 64 of the slotted tube 14. An atraumatic tip 90 in the form of a plug having a rounded end is provided at the second end 214 of the slotted tube 14. As illustrated, the atraumatic tip 90 may seal the second slotted tube opening 44 of the slotted tube 14.

FIG. 5B illustrates a cross sectional view of another exemplary embodiment of the distal portion of a slotted tube 14 with a balloon 16 in an unexpanded position. As illustrated, the slotted tube 14 includes a plurality of slots 18 positioned proximal to the proximal end 116 of balloon 16. Accordingly, a lumen liner 40 is provided on an inner surface 74 of lumen 24 extending over at least the plurality of slots 18 positioned proximal to the proximal end 116 of balloon 16. In one aspect, the lumen liner 40 extends only along the portion of the slotted tube 14 including slots 18. In another aspect, the lumen liner 40 extends to the proximal end 114 of the slotted tube 14. In aspects including a proximal tube 12, the lumen liner 40 may extend into or completely through the proximal lumen 22 of the proximal tube 12. One or more of the slots 18 positioned along the slotted tube 14 between the proximal end 116 and the distal end 216 of the balloon 16 are in fluid communication with a surface of the balloon 16 which forms expansion chamber 30 or may be expanded to form an expansion chamber 30 upon the introduction of an inflation media. The lumen liner 40 generally permits the communication of inflation media to the inflation chamber from a position proximal to the proximal end 116 of the balloon 16. An atraumatic tip 90 in the form of a coil 96 extending about a shaping ribbon 98 and including a rounded end is secured at the second end 214 of the slotted tube 14. As illustrated, the atraumatic tip 90 is secured within the second slotted tube opening 44 of the slotted tube 14 with an adhesive 80. Adhesive 80 may function to seal the second slotted tube opening 44 and direct inflation media through the slots 18 into the inflation chamber 30 of balloon 16.

FIG. 5C illustrates a cross sectional view of another exemplary embodiment of the distal portion of a slotted tube 14 with a balloon 16 in an unexpanded position. As illustrated, the slotted tube 14 includes a plurality of slots 18 positioned proximal to the proximal end of balloon 16. Accordingly, an inner tube 50 is provided within lumen 24 and is sealably secured to the inner surface 74 of the slotted tube 14 at a position distal to the proximal end 116 of the balloon 16. More generally, the inner tube 50 may be sealably secured at a location distal to the first slot 18 not covered by balloon 16. In one aspect, the inner tube 50 extends only along the portion of the slotted tube 14 including slots 18. In this aspect, a second end of the inner tube 50 is secured to an inner wall of the slotted tube at a location proximal to the most proximally positioned slot 18. In another aspect, the inner tube 50 extends to the proximal end 114 of the slotted tube 14. In this aspect, inflation media may be initially directed into a proximal end of the inflation lumen 60 without having to contact the inner surface 74 of slotted tube 14. In aspects including a proximal tube 12, the inner tube 50 may extend into or completely through the proximal lumen 22 of the proximal tube 12. In this aspect, inflation media may be initially directed into a proximal end of the inflation lumen 60 without having to contact the inner surface 72 of the proximal tube 12. The inner tube 50 generally extends over at least the plurality of slots 18 positioned proximal to the proximal end of balloon 16 and may be sealably secured to the inner surface 72 of the proximal tube 12 and the inner surface 74 of the slotted tube 14. One or more of the slots 18 positioned along the slotted tube 14 between the proximal end 116 and the distal end 216 of the balloon 16 are in fluid communication with a surface of the balloon 16 which forms expansion chamber 30 or may be expanded to form an expansion chamber 30 upon the introduction of an inflation media. An atraumatic tip 90 in the form of a coil 96 extending about a shaping ribbon 98 and including a rounded end is secured at the second end 214 of the slotted tube 14. As illustrated, the atraumatic tip 90 is secured within the second slotted tube opening 44 of the slotted tube 14 with an adhesive 80. Adhesive 80 may function to seal the second slotted tube opening 44 and direct inflation media through the slots 18 into the inflation chamber 30 of balloon 16.

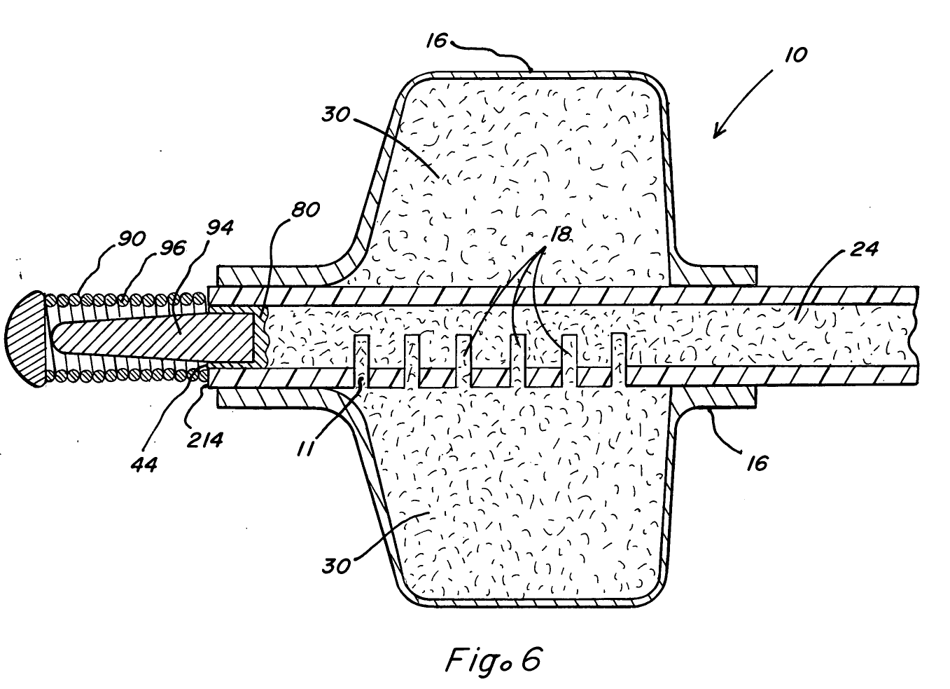

FIG. 6 illustrates a cross sectional view of another embodiment of the distal portion of a slotted tube 14 having an at least partially inflated balloon 16. The illustrated slotted tube 14 does not include any slots 18 positioned proximal to the proximal end 116 of balloon 16. The slots 18 extend along the slotted tube 14 between a proximal end 116 and a distal end 216 of the balloon 16. The slots 18 are in fluid communication with the expansion chamber 30 of balloon 16. The inflation media is provided through lumen 24 of slotted tube 14 to the inflation chamber 30 of the at least partially inflated balloon 16. As illustrated, the balloon 16 is configured to preferentially inflate to a desired diameter 326 sequentially along its length from a proximal end to a distal end of the balloon 16 as inflation media is introduced. Again, an atraumatic tip 90 in the form of a coil 96 extending about a shaping ribbon 98 and including a rounded end is secured at the second end 214 of the slotted tube 14. As illustrated, the atraumatic tip 90 is secured within the second slotted tube opening 44 of the slotted tube 14 with an adhesive 80. Adhesive 80 may function to seal the second slotted tube opening 44 and direct inflation media through the slots 18 into the inflation chamber 30 of balloon 16.

To use an occluding catheter 10 in accordance with the present invention, a user may insert the distal end of occluding catheter 10 into a bodily lumen of a patient using, for example, the Seldinger technique. The occluding catheter 10 is guided through the bodily lumen to a location within the patient requiring treatment. As occluding catheter 10 is guided through the patient, a user can manipulate the proximal tube 12 or the first end 114 of the slotted tube 14 to direct the second end 214 of the slotted tube 14 through the bodily lumen. When the second end 214 of the slotted tube 14 is positioned at or near the location within the bodily lumen requiring treatment, the user may initiate the desired treatment. In embodiments where the occluding catheter 10 includes a balloon 16 at or near the second end 214 of the slotted tube 14, the balloon 16 may be inflated to a desired size and/or pressure to affect the desired treatment. An occluding catheter 10 including a balloon 16, properly sized and configured, may enable a user to access more distal or tortuous regions of the body. For example, when the distal portion of the occluding catheter 10 has an outside diameter of around 0.014 inches, small lumen such as various arteries and veins in the brain and heart may be more easily accessed for diagnosis and/or treatment of the particular lumen or region.

Alternatively, after occluding catheter 10 is guided to a desired location within the body, occluding catheter 10 may be used to infuse fluid to that location. For example, fluids, such as saline solution, medicants, x-ray contrast liquids among other fluids, may be infused through second distal tube opening 44, and/or slots 18.

Occluding catheter 10 may further be used to guide surgical or diagnostic instruments over occluding catheter 10 to access a desired location in a bodily lumen. When the instrument is positioned at the desired location within the bodily lumen, at least one surgical or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. An occluding guidewire for accessing a bodily lumen of a patient, comprising:
a slotted tube having an outer surface and an inner surface, the inner surface defining a lumen extending between a first end and a second end of the slotted tube, the slotted tube further defining a plurality of slots extending between the outer surface of the slotted tube and the inner surface at a distal portion of the slotted tube to increase the flexibility of the distal portion of the slotted tube; and
a balloon having a first end and a second end and defining an inflation chamber, the balloon secured over the distal portion of the slotted tube with the inflation chamber in fluid communication with at least one of the plurality of slots.

2. An occluding guidewire, as in claim 1, further comprising a proximal tube defining a proximal lumen extending between a first end and a second end of the proximal tube, the second end of the proximal tube secured to a first end of the slotted tube with the proximal lumen of the proximal tube in fluid communication with the lumen of the slotted tube.

3. An occluding guidewire, as in claim 1, further comprising an inner tube having a first end and a second end, the inner tube defining an inflation lumen extending between a first end and a second end of the inner tube, the inner tube received within the lumen of the slotted tube, the second end of the inner tube sealingly secured to the inner wall of the slotted tube proximal to at least one slot and distal to a first end of the balloon, the inflation lumen in fluid communication with the at least one slot.

4. An occluding guidewire, as in claim 3, further comprising the second end of the inner tube sealingly secured to the inner wall of the slotted tube proximal to a proximal slot of the slotted tube.

5. An occluding guidewire, as in claim 4, further comprising a proximal tube defining a proximal lumen extending between a first end and a second end of the proximal tube, the second end of the proximal tube secured to a first end of the slotted tube with the proximal lumen of the proximal tube in fluid communication with the lumen of the slotted tube.

6. An occluding guidewire, as in claim 1, further comprising an inner lining secured to an inner wall of the slotted tube, the inner lining having a first end and a second end and defining an inflation lumen, the second end of the inner lining secured to the inner wall of the slotted tube proximal to at least one slot and distal to a first end of the balloon, the inflation lumen in fluid communication with the at least one slot.

7. An occluding guidewire, as in claim 6, further comprising the second end of the lumen liner secured to the inner wall of the slotted tube proximal to a proximal slot of the slotted tube.

8. An occluding guidewire, as in claim 7, further comprising a proximal tube defining a proximal lumen extending between a first end and a second end of the proximal tube, the second end of the proximal tube secured to a first end of the slotted tube with the proximal lumen of the proximal tube in fluid communication with the lumen of the slotted tube.
